(19) United States
(12) Patent Application Publication
(10) Pub. No.: US 2025/0356991 A1
(43) Pub. Date: Nov. 20, 2025

(54) DEVICE AND METHOD FOR X-RAY IMAGING, COMPUTER PROGRAM AND DATA MEDIUM

(71) Applicant: Siemens Healthineers AG, Forchheim (DE)

(72) Inventors: Joerg FREUDENBERGER, Kalchreuth (DE); Steffen KAPPLER, Hallerndorf-Pautzfeld (DE)

(73) Assignee: Siemens Healthineers AG, Forchheim (DE)

(21) Appl. No.: 19/208,940

(22) Filed: May 15, 2025

(30) Foreign Application Priority Data
May 16, 2024 (DE) 10 2024 204 540.3

(51) Int. Cl. G16H 30/20; G01N 23/04; G16H 30/40

(52) U.S. Cl. CPC G16H 30/20; G01N 23/04; G16H 30/40

(57) ABSTRACT

A device comprises a plurality of X-ray sources, an X-ray detector having at least one detector element, and a control device. The control device is configured to actuate the X-ray sources alternately, one after another, according to an actuation pattern to emit a respective X-ray pulse such that a respective X-ray source emits a X-ray pulses within the scope of the actuation pattern. The respective detector element is assigned a respective buffer memory and a respective data acquisition device. The data acquisition device is configured to write respective measurement data of the associated detector element or processing data determined from the respective measurement data repeatedly, synchronously with the actuation pattern, into the buffer memory such that the respective measurement data relates to X-ray radiation that is incident on the respective detector element due to a respective pulse of the X-ray pulses that is associated with the measurement data.

### CROSS-REFERENCE TO RELATED APPLICATION(S)

The present application claims priority under 35 U.S.C. § 119 to German Patent Application No. 10 2024 204 540.3, filed May 16, 2024, the entire contents of which are incorporated herein by reference.

### FIELD

One or more example embodiments of the present invention relate to a device for X-ray imaging comprising a plurality of X-ray sources, an X-ray detector having at least one detector element, and a control device. In addition, one or more example embodiments of the present relate to a method for X-ray imaging, a computer program and a data medium.

### BACKGROUND

In the field of X-ray imaging, both in the medical diagnostics domain and in other areas of application, for example for non-destructive testing of materials and for baggage scanners, system concepts are increasingly described in which a plurality of discrete or distributed X-ray sources are actuated sequentially in order to enable 3D imaging via projections from as many spatial directions as possible. There are various approaches for this, examples thereof being known by the names “static computed tomography”, “non-mechanical CT”, “fourth-generation CT” or “tomosynthesis with distributed sources”.

Stationary anodes are employed in most concepts that provide a multiplicity of sources since rotating anodes would be too expensive and too awkward to handle. With stationary anodes, however, the thermal load-bearing capacity is limited, which in turn limits the fields of application for tomography or tomosynthesis systems of said type.

Consequently, in the case of current, third-generation rotating computed tomography systems according to the prior art, a power output of 40 kW, for example, over an exposure interval of 200 us per projection scan can be emitted in order to achieve a sufficient number of X-ray quanta at the detector and hence an acceptable scan image quality. When stationary anodes are used, a similar number of X-ray quanta would also have to be captured at the detector in order to achieve a comparable scan image quality. However, due to the thermal load-bearing limits of stationary anodes, projection times in the region of several milliseconds would result in order to provide a sufficient X-ray dose since the stationary anodes can only draw approx. 2 to 5 kW of power over the exposure period without being damaged. The high exposure time restricts the potential uses of static computed tomography to applications without need for high temporal resolution. For example, CT scans of the thorax requiring a breathhold or a cardio CT would be possible, if at all, only to a very limited degree.

### SUMMARY

At least one object underlying one or more embodiments of the present invention is therefore to improve an achievable temporal resolution of an X-ray imaging scan, in particular using stationary anodes, and consequently, in particular, to open up further application areas for X-ray imaging using distributed stationary anodes as X-ray sources.

At least this object is achieved by an X-ray imaging device comprising a plurality of X-ray sources, an X-ray detector having at least one detector element, and a control device, wherein the control device is configured to drive the different X-ray sources alternately, one after another, in accordance with a predefined actuation pattern in order to emit a respective X-ray pulse such that the respective X-ray source emits a plurality of X-ray pulses within the scope of the actuation pattern, wherein a respective buffer memory and a respective data acquisition device are assigned to the respective detector element, wherein the data acquisition device is configured to write respective measurement data of the associated detector element or processing data determined from the respective measurement data repeatedly, synchronously in time with the actuation pattern, into the associated buffer memory such that the respective measurement data relates to X-ray radiation which is incident on the respective detector element due to a respective pulse of the X-ray pulses which is assigned to the measurement data.

One or more embodiments of the present invention are based on the idea of distributing the X-ray dose emitted by the individual X-ray source in the course of the imaging over multiple X-ray pulses spaced apart in time. In this regard it is possible to exploit the fact that a shorter continuous operating period or pulse duration enables a higher power to be used during the X-ray pulse. For example, given a reduction of the pulse time to a few microseconds, a stationary anode can be loaded with an electron beam power output of up to approx. 100 KW, as a result of which the requisite exposure time for the respective projection can be shortened by a factor of 20 to 50 compared to the use of a continuous X-ray pulse for said projection. Since the time between the emission of different X-ray pulses by one of the X-ray sources is used in order to emit X-ray pulses by the other X-ray sources, the time required for acquiring all of the projections can be significantly reduced or an applied X-ray dose can be increased as necessary for the same measurement time. The effect of the exposure time of an X-ray anode on the power that can be drawn by the same is discussed in general terms already in S. Bartzsch, U. Oelfke, Line focus x-ray tubes-a new concept to produce high brilliance x-rays, 2017 Phys. Med. Biol. 62 8600.

An X-ray dose that is to be emitted can therefore be distributed over multiple, in particular ultrashort, X-ray pulses, spaced apart in time, of, for example, a few microseconds or even less than 1000 nanoseconds, wherein the X-ray sources used are rotated, in particular cyclically, so that the emitted radiation is applied in each case by way of a cold emitter, i.e. via an X-ray source whose anode temperature in particular substantially corresponds to the ambient temperature. Since X-ray quanta originating from different ones of the X-ray sources are therefore detected at the detector element at different times, the detector elements, i.e. individual pixels of a planar X-ray detector, for example, are read out at a sufficiently high frame rate, which can be realized through the use of separate buffer memories and data acquisition devices for the individual detector elements.

The X-ray detector may in particular comprise a plurality of detector elements or pixels in order to detect a respective X-ray intensity which is incident locally on the respective detector element. For example, each detector element can form one detector pixel of a one-dimensional or two-dimensional array of detector pixels. The detector elements may in particular be counting X-ray detectors by which the incidence of individual X-ray quanta is detected and counted. Alternatively, however, it is also possible for example to use scintillator-based detector elements, in which case, for example, a scintillator without afterglow or with a very short afterglow time may be used in conjunction with a silicon photomultiplier in order to achieve short readout times and a good separation of the measurement data for different X-ray pulses.

The data acquisition device is preferably configured or synchronized with the control device in such a way that the writing of the measurement data or the processing data into the buffer memory for the respective X-ray pulse is completed before the next X-ray pulse is emitted.

The number of X-ray sources may be predetermined in particular by the number of projection geometries to be acquired. For example, 15-30 X-ray sources and therefore projection geometries may be sufficient for a tomosynthesis application, whereas several 100 or even 1000 or more projection geometries and therefore X-ray sources may be used for a computed tomography scan, for example. Within the scope of the actuation pattern, the respective X-ray source can be actuated for example more than one hundred times or more than 500 times, for example approximately 1000 times, in order to emit a respective X-ray pulse. By this means or mechanism, the duration of the individual X-ray pulse and consequently the loadbearing time of the respective X-ray source can be reduced accordingly.

The X-ray source may in particular comprise a stationary anode and/or a field-effect transmitter, based on nanotubes for example. By a stationary anode is understood an anode which is arranged rigidly relative to the cathode and in particular does not rotate. Stationary anodes can be constructed considerably smaller and more easily than rotating anodes. Results consistent with real-world practice are achieved for example also at electrical focal spot sizes of approx. 10 mm².

As will be explained in more detail later, a preprocessing of the data of the individual detector element can be performed already in the respective detector element itself or in its associated data acquisition device, in particular in order to combine the measurement data for the different X-ray pulses of the same X-ray source, for example by a summation. By this means or mechanism, there results substantially the same volume of data as would result when using a single, continuous X-ray pulse for the respective projection geometry. Unnecessarily large volumes of data or, as the case may be, data transmission requirements between the detector elements and the control device or an evaluation device are therefore avoided as a result.

In principle, the time synchronization of the readout of the detector element with the actuation pattern can be realized via a self-synchronization, for example by detection of measurement signal edges. Preferably, however, the control device and the different detector elements are synchronized explicitly, for example via a common clock signal or through control or triggering of the detector elements by the control device.

The control device is preferably configured to actuate the respective X-ray source in order to emit a respective X-ray pulse having a pulse length of less than 300 μs or less than 100 μs or less than 10 μs and/or to actuate successive X-ray sources in the actuation pattern one after the other in a time interval of less than 300 μs or less than 100 μs or less than 10 μs.

As has already been described above, by increasingly shortening the pulse duration it is possible to use higher and higher energies, as a result of which the required total exposure time can be reduced. By using separate buffer memories per detector element, a time required for writing the measurement data or the processing data for the respective X-ray pulse can be minimized, as a result of which X-ray pulses for different X-ray sources can be emitted one after another in quick succession and nonetheless be separated on the detector side. This enables the requisite measurement time to be shortened further or the temporal resolution of the measurement to be further improved.

The respective data acquisition device can be configured to store the measurement data associated with the separate X-ray pulses or the processing data determined from the respective measurement data in separate memory areas of the buffer memory and, following termination of the actuation pattern, to link the measurement data and/or the processing data stored in the buffer memory and associated with the respective X-ray source to one another via an arithmetic operation in order to provide overall data associated with the respective X-ray source.

In the simplest case, the individual sets of measurement data or processing data are added, for example in order to add the photon counts, charges or intensities recorded for the separate X-ray pulses. Depending on the actual type of data acquisition, however, a multiplication, a division or a subtraction may also be beneficial.

Through the combination of the measurement data or processing data of all of the X-ray pulses of the same X-ray source, the overall data can in particular substantially correspond to the result that would be expected when using a continuous longer X-ray pulse of the same intensity. By combining the measurement data or processing data to produce the overall data it is possible in particular to achieve a considerable reduction in the volume of data that must be transferred from the respective data acquisition device to a processing device or to the control device. Accordingly, in particular communication paths and processing approaches which are inherently designed for using continuous X-ray pulses of the individual X-ray sources can also be used for the alternating, pulsewise use of the individual X-ray sources according to one or more embodiments of the present invention. The approach according to one or more embodiments of the present invention can therefore be implemented with little overhead.

Alternatively, the respective data acquisition device can be configured to store, for a respective X-ray source, respective first data of the measurement data associated in each case with the first X-ray pulse of the respective X-ray source in the actuation pattern, or processing data determined from the first measurement data, in one of the memory areas of the buffer memory associated with the respective X-ray source and respective further measurement data associated with an X-ray pulse of the respective X-ray source following the first X-ray pulse in the actuation pattern, or to link provisional processing data determined from the further measurement data to the previous contents of the memory area of the buffer memory associated with the respective X-ray source via an arithmetic operation and to store the result of the linking as processing data in the memory area of the buffer memory associated with the respective X-ray source.

The determining of the further measurement data and the linking of the further measurement data or of the respective further provisional processing result to the previous contents of the memory area of the buffer memory associated with the respective X-ray source can be repeated in particular for each X-ray pulse of the respective X-ray source after the first X-ray pulse.

Following termination of the actuation pattern, overall data is therefore present in the memory area associated with the respective X-ray source for each of the X-ray sources which result from an arithmetic operation linking all of the measurement data associated with X-ray pulses of the respective X-ray source or, as the case may be, correspond to their provisional processing data. The ultimately determined overall data therefore corresponds to the above-discussed overall data resulting in the case of a downstream consolidation of the measurement data or processing data. Compared to the above-explained approach, however, considerably smaller buffer memories can be used in this case. On the other hand, the above-explained subsequent data consolidation can be advantageous in order to defer the amount of time required for consolidating the measurement data or processing data to a time interval following termination of the actuation pattern, and therefore following the measurement operation, and consequently potentially shorten the duration of the measurement.

The memory areas can also be assigned dynamically to the X-ray sources for a respective playing-out of the actuation pattern. For example, when a cyclical buffer memory is used, a pointer which describes the position of the respective memory access can have different values at the start of separate iterations of the actuation pattern, which can lead to a different assignment of the memory areas to the X-ray sources.

The buffer memory can be embodied in particular as a cyclical buffer, a read and write position of the buffer memory being specified via a cyclical memory pointer. In this case the data acquisition device can be configured to increment the memory pointer by a predefined value both after the respective first measurement data or the processing data determined therefrom is stored and after the respective linkage result is stored, wherein the cycle length of the cyclical memory pointer corresponds to the product from the predefined value and the number of X-ray sources.

In other words, the cycle length of the cyclical memory pointer is chosen such that when n X-ray sources are used, the memory pointer again points in each case to the same memory cell or address of the buffer memory after n storage operations. Thus, if the X-ray sources are repeatedly actuated in a fixed order, the described cycle length of the cyclical memory pointer leads during the acquisition of the respective measurement data to the memory pointer always pointing to the memory area assigned to the X-ray source last used for emitting an X-ray pulse. The memory pointer can therefore be used both for reading out the previous contents of the memory area associated with the respective X-ray source and for storing the linkage result. A particularly simple and robust synchronization of the data acquisition with the emission of the X-ray pulses can be achieved by this means or mechanism.

However, using a cyclical buffer memory may also be advantageous when the measurement data or processing data for the individual X-ray pulses is initially written into separate memory areas of the buffer memory and is combined only following termination of the actuation pattern for a transmission or else is transferred separately to an evaluation device. In this case the buffer memory should be chosen sufficiently large so that data acquired within the scope of the actuation pattern is not overwritten within the same actuation pattern, but is overwritten only at a later time, for example during a subsequent data acquisition when a further actuation pattern is played out.

The respective detector element is preferably formed via a photon-counting X-ray detector or a respective pixel of a spatially resolved photon-counting X-ray detector forming a plurality of or all of the detector elements. Photon-counting X-ray detectors or their pixels enable individual incoming photons to be detected and counted and consequently possess in particular a sufficient temporal resolution to acquire well-separated measurement data for each individual X-ray pulse, even with very short X-ray pulses. In this case the measurement data can in particular indicate the respective number of photons detected by the respective detector element since the emission of the respective X-ray pulse.

As an alternative to a photon-counting X-ray detector, a detector having a scintillator without afterglow or with a sufficiently short afterglow time could be used, for example. A sufficiently quick readout can be achieved in this case by use of a silicon photomultiplier, for example.

The respective X-ray source may comprise a stationary anode and/or a cathode formed by a field-effect emitter. Using a field-effect emitter as a cathode enables rapid activation and deactivation of electron emission and consequently short X-ray pulses. Field-effect emitters based on carbon nanotubes can be used, for example.

Using the different X-ray sources in alternation enables these to cool down between the emission of the individual X-ray pulses. High X-ray energies can therefore be realized also by a stationary anode. The device according to one or more embodiments of the present invention can therefore provide high X-ray power outputs with little technical overhead and consequently at low cost.

The device according to one or more embodiments of the present invention may be for example a computed tomography system or a tomosynthesis system for imaging at least one examination region of a patient in the course of a medical imaging procedure. Although the device described, as already explained in the introduction, may also be designed for example for materials testing or as a baggage scanner, the explained embodiment is particularly advantageous in the field of medical imaging because in that context, as a result of short examination times, not only can the utilization of the device be improved, but artifact formation due to patient movement during the imaging can also be minimized.

In a computed tomography system, the X-ray sources can be disposed in a distributed arrangement around the patient, in particular over an angle of 360°, in which case, for example, an arrangement in one or more rings and also a helix-shaped arrangement around the patient are possible. In order to achieve a high spatial resolution, at least 500 or at least 1000 X-ray sources, for example, can be used in a computed tomography system.

For a tomosynthesis system, it may be sufficient to employ a linear arrangement of X-ray sources or an arrangement over a certain solid angle segment of, for example, at least 40° or at least 60°. For tomosynthesis applications, it may be sufficient to use 15-25 X-ray sources. The tomosynthesis system can be used for breast tomosynthesis, for example.

In particular, at least two of the X-ray sources may be separated from one another by a distance of more than 30 cm or more than 50 cm or more than 1 m. In addition or alternatively, the X-ray sources may be disposed in a distributed arrangement over an angular range of at least 15° or at least 30° or at least 60° or at least 90° relative to an isocenter of the imaging. The X-ray sources may in particular be arranged at the same distance or angular distance from one another.

The spaced-apart arrangement of the X-ray sources or their distribution over a certain angular range leads to a different acquisition geometry resulting, depending on by which of the X-ray sources the respective X-ray pulse is emitted. The use of separate X-ray sources which are actuated one after the other therefore serves in this case not only for improving performance but also for providing different acquisition geometries.

In addition to the device according to embodiments of the present invention, one or more embodiments of the present invention also relate to a method for X-ray imaging by a device comprising a plurality of X-ray sources and an X-ray detector having at least one detector element, wherein the different X-ray sources are actuated in alternation, one after the other, in accordance with a predefined actuation pattern for the purpose of emitting a respective X-ray pulse such that the respective X-ray source emits a number of X-ray pulses in the course of the actuation pattern, wherein respective measurement data of the respective detector element or processing data determined from the respective measurement data is written repeatedly, synchronously in time with the actuation pattern, into a buffer memory associated with the respective detector element such that the respective measurement data relates to X-ray radiation which is incident on the respective detector element due to a respective pulse of the X-ray pulses which is associated with the measurement data.

The method according to embodiments of the present invention can be implemented in particular via the device according to embodiments of the present invention. Independently thereof, features disclosed in relation to the device according to embodiments of the present invention can be applied together with the advantages cited there to the method according to embodiments of the present invention, and vice versa.

One or more embodiments of the present invention further relate to a computer program comprising instructions which are configured to perform the method according to embodiments of the present invention when the computer program is executed on a device comprising a plurality of X-ray sources and an X-ray detector having at least one detector element.

In addition, one or more embodiments of the present invention relate to a non-transitory data medium or computer-readable medium which comprises the computer program according to embodiments of the present invention. The computer program according to embodiments of the present invention or the data medium according to embodiments of the present invention can be developed via the features explained in relation to the method according to embodiments of the present invention or in relation to the device according to embodiments of the present invention together with the advantages cited there, and vice versa.

### BRIEF DESCRIPTION OF THE DRAWINGS

Further advantages and specific details of the present invention will become apparent from the following exemplary embodiments as well as from the associated schematic drawings, in which:

FIG. 1 shows an exemplary embodiment of a device for X-ray imaging according to the present invention, and

FIG. 2 shows a flowchart of an exemplary embodiment of the method for X-ray imaging according to the present invention.

### DETAILED DESCRIPTION

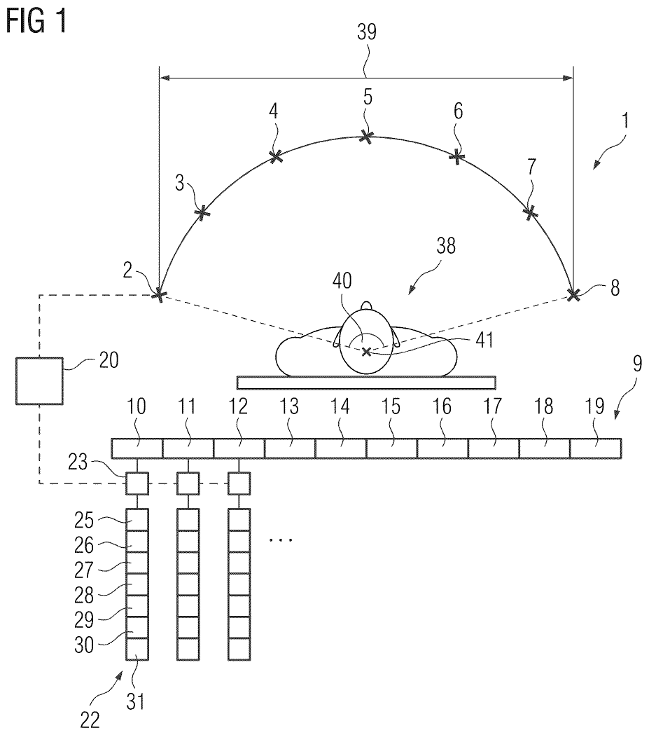

FIG. 1 shows a device 1 for X-ray imaging comprising a plurality of X-ray sources 2 to 8 and an X-ray detector 9 having a plurality of detector elements 10 to 19 or pixels. In the example, the device 1 is a tomosynthesis system in which the X-ray sources 2-8 are distributed over an angular range 40 of approx. 70° relative to an isocenter 41 of the imaging and consequently are arranged at a relatively great distance 39 from one another. As has already been explained in the general part, tomosynthesis imaging would also be possible given a distribution of the X-ray sources over a narrower angular range of, for example, 15°. For clarity of illustration reasons, only 7 X-ray sources 2-8 and 10 detector elements 10 to 19 are shown in the example. In a real-world implementation, 15-30 X-ray sources and a large number of pixels or detector elements, for example an array composed of 256×256 detector elements, are typically used in tomosynthesis systems.

In an alternative embodiment, a computed tomography system for static computed tomography could be provided by distributing the X-ray sources 2 to 8 around the entire patient 38, for example in several rings or along a helix.

By irradiating the patient 38 with the different X-ray sources 2 to 8 and acquiring respective image data via the X-ray detector 9 there result projection images acquired from different perspectives, from which three-dimensional image data can be reconstructed in a known manner, for example via the control device 20 of the device 1 or via a separate evaluation device (not shown).

When X-ray sources are deployed in a distributed arrangement, X-ray sources having stationary anodes, i.e. having rigid anodes, should generally be used for installation space and cost reasons. As has already been explained in the general part of the description, relatively long measurement times result for stationary anodes due to the limiting of the permitted power input into the anode when a conventional measurement protocol is used in which the patient 38 is irradiated with a continuous X-ray pulse whose length is chosen in such a way that an adequate X-ray dose is achieved at the detector, which means that certain types of measurements are not possible or at least a significant risk of a disruption of the measurement results due to motion artifacts.

In order to allow the use of higher X-ray energies and consequently shorter exposure times, a modified approach is therefore used in the device 1. In this case the control device 20 actuates the different X-ray sources 2 to 8 in alternation, one after the other, in accordance with a predefined actuation pattern in order to emit a respective X-ray pulse 21 such that the respective X-ray source 2 to 8 emits a plurality of X-ray pulses 21, spaced apart in time, within the scope of the actuation pattern. Thus, instead of a long, continuous X-ray pulse, a number of short X-ray pulses of the respective X-ray source 2 to 8 are used, wherein in the pauses between the operation of the respective X-ray source 2 to 8, during which the respective X-ray source 2 to 8 cools down, X-ray pulses are emitted by the other of the X-ray sources 2 to 8.

In order to allow a separation of the measurement data for the different X-ray pulses, a separate buffer memory 22 and a separate data acquisition device 23 are used for each of the detector elements 10 to 19. The data acquisition device 23 is in this case configured to write respective measurement data 24 of the associated detector element 10 to 19 or processing data 42 determined therefrom repeatedly, synchronously in time with the actuation pattern, into the associated buffer memory 22.

The time synchronization can be achieved for example via a common clock signal for the control device 20 and the data acquisition devices 23, or also by direct actuation of the data acquisition devices 23 by the control device, for example via a trigger signal. It is achieved by this means or mechanism and by using relatively rapidly switching X-ray sources 2 to 8, for example by the use of a field-effect emitter as cathode, that the respective measurement data 24 relates to that X-ray radiation which is incident on the respective detector element 10 to 19 due to a respective pulse of the X-ray pulses 21 which is associated with the measurement data 24.

In the following it is assumed by way of example that the detector elements 10 to 19 are photon-counting X-ray detectors, whereby a number of photons detected at the respective detector element 10 to 19 since the start of the emission of the last emitted X-ray pulse are acquired in each case as respective measurement data 24.

By using sufficiently short X-ray pulses, for example having a pulse length of less than 10 μs, a high X-ray power can also be used with stationary anodes and consequently a short measurement time can be achieved overall.

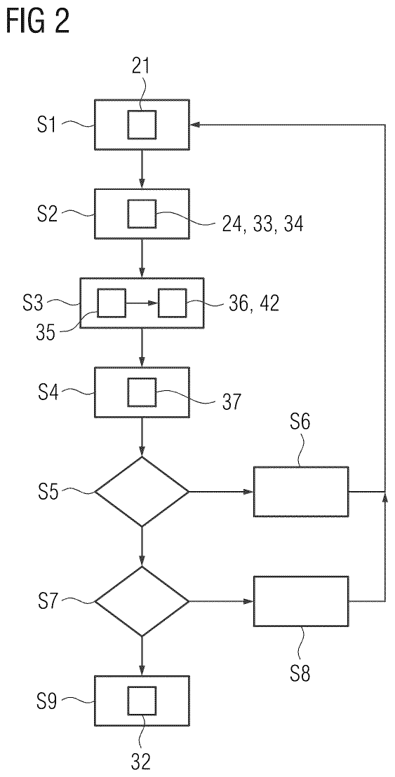

A method for X-ray imaging implemented by the device 1 is explained by way of example below in more detail with reference to the flowchart shown in FIG. 2. In this example, steps S1 to S4 are performed once for each of the X-ray pulses 21 that are to be emitted. After each of the passes, a changeover is then performed in step S6 or S8, as will be explained in more detail later, to switch the X-ray source 2 to 8 actuated in each case for the purpose of emitting the X-ray pulse 21 such that the X-ray sources 2 to 8 are actuated in alternation in accordance with a predefined actuation pattern, wherein each of the X-ray sources 2 to 8 is actuated multiple times for the purpose of emitting a respective X-ray pulse 21.

In step S1, initially in each iteration of steps S1 to S4, the currently selected one of the X-ray sources 2 to 8 is actuated by the control device 20 in order to emit a short X-ray pulse 21. In the first iteration of steps S1 to S4, the first X-ray source 2 is therefore actuated in step S1, the second X-ray source 3 in the next iteration, etc. As of the eighth iteration, i.e. following the actuation of the seventh X-ray source 8, the preceding sequence is repeated, i.e. initially, once again, the first X-ray source 2 is actuated, then the second X-ray source 3, etc.

In step S2, the measurement data 24, i.e., in the example, the number of X-ray photons detected since the start of the X-ray pulse, is read out from the respective detector element 10 to 19 for each of the detector elements 10 to 19 by the respective associated data acquisition device 23.

For the first measurement data 33 acquired during the first seven iterations of steps S1 to S4, which data is associated in each case with a first X-ray pulse 21 of the respective X-ray source 2 to 9 in the actuation pattern, the respective first measurement data 33 is written in step S3 directly into a memory area 25 to 31 of the respective buffer memory 22 associated with the respective X-ray source 2 to 8.

This is realized in the example in that a write access is performed to a memory address specified by a cyclical memory pointer 37, wherein the memory pointer 37 is incremented by a predefined value in the following step S4, i.e. after the respective write operation, in order to select the memory area 25 to 31 associated with the respective next of the X-ray sources 2-8.

In step S5, a check is carried out in each case to determine whether the last X-ray source 8 is currently already selected and consequently has already emitted an X-ray pulse 21. If this is not the case, then the next of the X-ray sources 2-8 is selected in each case in step S6 and the method is repeated starting from step S1.

If, on the other hand, the X-ray pulse 21 was lastly emitted by the last X-ray source 8, then it is checked in step S7 whether the individual X-ray sources 2 to 8 have already emitted a sufficient number of X-ray pulses 21. If this is not the case, then the first X-ray source 2 is selected once again in step S8 and the method is continued from step S1 for the purpose of emitting a further X-ray pulse 21 by said X-ray sources 2.

After the seventh iteration of steps S1 to S4, the respective memory area 25 to 31 of the respective buffer memory 22 therefore comprises the respective first measurement data 33 relating to the first X-ray pulse 21 of the respective X-ray source 2 to 8. In the example, the cycle length of the memory pointer 37 is chosen such that it corresponds to the product from the predefined value of the increment in step S4 and the number of X-ray sources 2 to 8. Accordingly, the seventh increment of the memory pointer 37 leads, after the writing of the first measurement data 33 for the first X-ray pulse 21 of the seventh X-ray source 8 in the seventh iteration of step S4, to the memory pointer 37 again pointing to the respective memory area 25 associated with the first X-ray source 2.

As of the eighth repetition of steps S1 to S4, the further measurement data 34 acquired previously in step 2 is therefore not written immediately into the respective memory area 25 to 31 in step S3, but instead is added to the previous contents 35 of the memory area 25 to 31 of the buffer memory 22 associated with the respective X-ray source 2 to 8, whereupon the linkage result 36 of this addition is written as processing data 42 into the respective memory area 25 to 31. The memory area for reading and writing is selected by the memory pointer 37.

The procedure described leads to all the measurement results 24 of the respective detector element 10 to 19 which are associated with the different X-ray pulses 21 of the same X-ray source 2 to 8 being added to one another. Accordingly, following termination of the actuation pattern, i.e. when it is detected in step S7 that all the X-ray pulses 21 to be emitted have already been emitted, the contents 35 of the memory area 25 to 31 associated with the respective X-ray source 2 to 8 are provided in step S9 as overall data 32 for the respective X-ray source. The overall data can therefore substantially correspond to that data that would have been acquired if a continuous, long X-ray pulse were to have been emitted by the respective X-ray source 2 to 8.

The example discussed in the foregoing is based on the premise that the addition or, as the case may be, generally the arithmetic operation linking the different measurement data 24 for pulses of the same X-ray source 2 to 8 is performed immediately in the course of the data acquisition. Alternatively, however, it would be possible to use a larger buffer memory 22 in which each of the X-ray pulses 21 emitted by the different X-ray sources 2 to 8 is assigned a respective memory area in which the measurement data 24 associated with said X-ray pulse 21 can be stored. In this case, following termination of the actuation pattern, the measurement data 24 stored in the buffer memory 22 and associated with the respective X-ray source 2 to 8 can be linked with one another via an arithmetic operation, i.e., for example, can be added in order to provide overall data 32 associated with the respective X-ray source 2 to 8.

Alternatively, it would also be possible to transfer the measurement data 24 or respective processing data determined therefrom that is stored in the buffer memory 22 separately to the control device 20 or a separate evaluation device. However, the previously explained consolidation of measurement data 24 of different X-ray pulses 21 of the same X-ray source 2 to 8 may be advantageous in order to minimize the transmission time or the necessary transmission bandwidth.

The explained method or the explained device can be implemented in particular by execution of a suitable computer program by the device 1. The instructions of the computer program can in this case be performed in particular partly on the control device 20 and partly on the individual data acquisition devices 23.

Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A device for X-ray imaging, the device comprising:
a plurality of X-ray sources;
an X-ray detector having at least one detector element; and
a control device configured to actuate the plurality of X-ray sources alternately, one after another, in accordance with an actuation pattern to emit a respective X-ray pulse such that a respective X-ray source emits a number of X-ray pulses within the scope of the actuation pattern, wherein
a respective buffer memory and a respective data acquisition device are assigned to a respective detector element among the at least one detector element, and
the respective data acquisition device is configured to write, into the respective buffer memory, respective measurement data of the respective detector element or processing data determined from the respective measurement data repeatedly, synchronously in time with the actuation pattern, such that the respective measurement data relates to X-ray radiation that is incident on the respective detector element due to a respective pulse, of the number of X-ray pulses, that is associated with the respective measurement data.

2. The device as claimed in claim 1, wherein the control device is configured to at least one of
actuate the respective X-ray source to emit the respective X-ray pulse having a pulse length of less than 300 μs, or
actuate X-ray sources, of the plurality of X-ray sources, succeeding one another in the actuation pattern in a time interval of less than 300 μs.

3. The device as claimed in claim 1, wherein the respective data acquisition device is configured to
store the respective measurement data associated with X-ray pulses or the processing data determined from the respective measurement data in separate memory areas of the respective buffer memory, and
following termination of the actuation pattern, link at least one of the respective measurement data or the processing data stored in the respective buffer memory and associated with the respective X-ray source with one another via an arithmetic operation to provide overall data associated with the respective X-ray source.

4. The device as claimed in claim 1, wherein the respective data acquisition device is configured to
store, for the respective X-ray source, first measurement data associated with a first X-ray pulse in each case of the respective X-ray source in the actuation pattern, or processing data determined from the first measurement data, in a memory area of the respective buffer memory,
link respective further measurement data associated with an X-ray pulse of the respective X-ray source following the first X-ray pulse in the actuation pattern, or provisional processing data determined from the respective further measurement data, via an arithmetic operation, to previous contents of the memory area of the respective buffer memory, and
store a linking result as processing data in the memory area of the respective buffer memory.

5. The device as claimed in claim 4, wherein
the respective buffer memory is a cyclical buffer,
a read and write position of the respective buffer memory is specified via a cyclical memory pointer,
the respective data acquisition device is configured to increment the cyclical memory pointer by a value after the first measurement data or the processing data determined from the first measurement data is stored and after a respective linkage result is stored, and
a cycle length of the cyclical memory pointer corresponds to a product of the value and a number of the plurality of X-ray sources.

6. The device as claimed in claim 1, wherein the respective detector element is a photon-counting X-ray detector or a respective pixel of a spatially resolved photon-counting X-ray detector forming a plurality of or all detector elements.

7. The device as claimed in claim 1, wherein the respective X-ray source comprises at least one of a stationary anode or a cathode formed by a field-effect emitter.

8. The device as claimed in claim 1, wherein the device is a computed tomography system or a tomosynthesis system for imaging at least one examination region of a patient during a medical imaging procedure.

9. The device as claimed in claim 1, wherein at least one of
at least two of the plurality of X-ray sources are separated from one another by a distance of more than 30 cm, or
the plurality of X-ray sources are disposed in a distributed arrangement over an angular range of at least 15° relative to an isocenter of the X-ray imaging.

10. A method for X-ray imaging via a device including a plurality of X-ray sources and an X-ray detector having at least one detector element, the method comprising:
alternately actuating the plurality of X-ray sources one after another in accordance with an actuation pattern to emit a respective X-ray pulse such that a respective X-ray source emits a plurality of X-ray pulses within a scope of the actuation pattern; and
repeatedly writing respective measurement data of a respective detector element or processing data determined from the respective measurement data, synchronously in time with the actuation pattern, into a buffer memory associated with the respective detector element such that the respective measurement data relates to X-ray radiation that is incident on the respective detector element due to a respective pulse of the plurality of X-ray pulses, which is associated with the respective measurement data.

11. A non-transitory computer-readable storage medium storing computer-executable instructions that, when executed on a device including a plurality of X-ray sources and an X-ray detector having at least one detector element, cause the device to perform the method of claim 10.

12. The device as claimed in claim 1, wherein at least one of
at least two of the plurality of X-ray sources are separated from one another by a distance of more than 50 cm, or
the plurality of X-ray sources are disposed in a distributed arrangement over an angular range of at least 30° relative to an isocenter of the X-ray imaging.

13. The device as claimed in claim 1, wherein at least one of
at least two of the plurality of X-ray sources are separated from one another by a distance of more than more than 1 m, or
the plurality of X-ray sources are disposed in a distributed arrangement over an angular range of at least 90° relative to an isocenter of the X-ray imaging.

14. The device as claimed in claim 1, wherein the control device is configured to at least one of
actuate the respective X-ray source to emit the respective X-ray pulse having a pulse length of less than 100 μs, or
actuate X-ray sources, of the plurality of X-ray sources, succeeding one another in the actuation pattern in a time interval of less than 100 μs.

15. The device as claimed in claim 1, wherein the control device is configured to at least one of
actuate the respective X-ray source to emit the respective X-ray pulse having a pulse length of less than 10 μs, or
actuate X-ray sources, of the plurality of X-ray sources, succeeding one another in the actuation pattern in a time interval of less than 10 μs.

16. The device as claimed in claim 2, wherein the respective data acquisition device is configured to
store the respective measurement data associated with X-ray pulses or the processing data determined from the respective measurement data in separate memory areas of the respective buffer memory, and
following termination of the actuation pattern, link at least one of the respective measurement data or the processing data stored in the respective buffer memory and associated with the respective X-ray source with one another via an arithmetic operation to provide overall data associated with the respective X-ray source.

17. The device as claimed in claim 2, wherein the respective data acquisition device is configured to
store, for the respective X-ray source, first measurement data associated with a first X-ray pulse in each case of the respective X-ray source in the actuation pattern, or processing data determined from the first measurement data, in a memory area of the respective buffer memory,
link respective further measurement data associated with an X-ray pulse of the respective X-ray source following the first X-ray pulse in the actuation pattern, or provisional processing data determined from the respective further measurement data, via an arithmetic operation, to previous contents of the memory area of the respective buffer memory, and
store a linking result as processing data in the memory area of the respective buffer memory.

18. The device as claimed in claim 3, wherein at least one of
at least two of the plurality of X-ray sources are separated from one another by a distance of more than 30 cm, or
the plurality of X-ray sources are disposed in a distributed arrangement over an angular range of at least 15° relative to an isocenter of the X-ray imaging.

19. The device as claimed in claim 4, wherein at least one of
at least two of the plurality of X-ray sources are separated from one another by a distance of more than 30 cm, or
the plurality of X-ray sources are disposed in a distributed arrangement over an angular range of at least 15° relative to an isocenter of the X-ray imaging.
